(19) United States
(12) Patent Application Publication
(10) Pub. No.: US 2017/0079833 A1
(43) Pub. Date: Mar. 23, 2017

(54) TRANSCUTANEOUS TREATMENT SYSTEMS, COOLING DEVICES, AND METHODS FOR COOLING NERVES

(71) Applicant: Zeltiq Aesthestics, Inc., Pleasanton, CA (US)

(72) Inventors: George Frangineas, JR., Fremont, CA (US); Leonard C. DeBenedictis, Dublin, CA (US)

(21) Appl. No.: 15/271,121

(22) Filed: Sep. 20, 2016

Related U.S. Application Data
(60) Provisional application No. 62/221,490, filed on Sep. 21, 2015.

(51) Int. Cl. A61F 7/00; A61B 5/00

(52) U.S. Cl. CPC A61F 7/007; A61B 5/4893; A61F 2007/0075; A61F 2007/0056; A61F 2007/0087

(57) ABSTRACT

Treatment systems, methods, and apparatuses for improving the appearance of skin and other treatments are described. Aspects of the technology are directed to improving the appearance of skin by transcutaneously cooling and affecting nerve tissue so as to inhibit facial muscular contractions and thereby reduce dynamic wrinkling. A non-invasive nerve cooling device can be applied to the subject's head to attenuate nervous system signals to facial muscles.

### CROSS-REFERENCE TO RELATED APPLICATION

The present application claims the benefit of and priority under 35 U.S.C. §119(e) to U.S. Provisional Patent Application No. 62/221,490, filed Sep. 21, 2015, which is incorporated herein by reference in its entirety.

### INCORPORATION BY REFERENCE OF COMMONLY-OWNED APPLICATIONS AND PATENTS

The following commonly assigned U.S. Patent Applications and U.S. Patents are incorporated herein by reference in their entirety:

U.S. Patent Publication No. 2008/0287839 entitled “METHOD OF ENHANCED REMOVAL OF HEAT FROM SUBCUTANEOUS LIPID-RICH CELLS AND TREATMENT APPARATUS HAVING AN ACTUATOR”;

U.S. Pat. No. 6,032,675 entitled “FREEZING METHOD FOR CONTROLLED REMOVAL OF FATTY TISSUE BY LIPOSUCTION”;

U.S. Patent Publication No. 2007/0255362 entitled “CRYOPROTECTANT FOR USE WITH A TREATMENT DEVICE FOR IMPROVED COOLING OF SUBCUTANEOUS LIPID-RICH CELLS”;

U.S. Pat. No. 7,854,754 entitled “COOLING DEVICE FOR REMOVING HEAT FROM SUBCUTANEOUS LIPID-RICH CELLS”;

U.S. Pat. No. 8,337,539 entitled “COOLING DEVICE FOR REMOVING HEAT FROM SUBCUTANEOUS LIPID-RICH CELLS”;

U.S. Patent Publication No. 2008/0077201 entitled “COOLING DEVICES WITH FLEXIBLE SENSORS”;

U.S. Pat. No. 9,132,031 entitled “COOLING DEVICE HAVING A PLURALITY OF CONTROLLABLE COOLING ELEMENTS TO PROVIDE A PREDETERMINED COOLING PROFILE”;

U.S. Patent Publication No. 2009/0118722, filed Oct. 31, 2007, entitled “METHOD AND APPARATUS FOR COOLING SUBCUTANEOUS LIPID-RICH CELLS OR TISSUE”;

U.S. Patent Publication No. 2009/0018624 entitled “LIMITING USE OF DISPOSABLE SYSTEM PATIENT PROTECTION DEVICES”;

U.S. Patent Publication No. 2009/0018623 entitled “SYSTEM FOR TREATING LIPID-RICH REGIONS”;

U.S. Patent Publication No. 2009/0018625 entitled “MANAGING SYSTEM TEMPERATURE TO REMOVE HEAT FROM LIPID-RICH REGIONS”;

U.S. Patent Publication No. 2009/0018627 entitled “SECURE SYSTEM FOR REMOVING HEAT FROM LIPID-RICH REGIONS”;

U.S. Patent Publication No. 2009/0018626 entitled “USER INTERFACES FOR A SYSTEM THAT REMOVES HEAT FROM LIPID-RICH REGIONS”;

U.S. Patent No. 6,041,787 entitled “USE OF CRYOPROTECTIVE AGENT COMPOUNDS DURING CRYOSURGERY”;

U.S. Patent No. 8,285,390 entitled “MONITORING THE COOLING OF SUBCUTANEOUS LIPID-RICH CELLS, SUCH AS THE COOLING OF ADIPOSE TISSUE”;

U.S. Provisional Patent Application Ser. No. 60/941,567 entitled “METHODS, APPARATUSES AND SYSTEMS FOR COOLING THE SKIN AND SUBCUTANEOUS TISSUE”;

U.S. Pat. No. 8,275,442 entitled “TREATMENT PLANNING SYSTEMS AND METHODS FOR BODY CONTOURING APPLICATIONS”;

U.S. patent application Ser. No. 12/275,002 entitled “APPARATUS WITH HYDROPHILIC RESERVOIRS FOR COOLING SUBCUTANEOUS LIPID-RICH CELLS”;

U.S. patent application Ser. No. 12/275,014 entitled “APPARATUS WITH HYDROPHOBIC FILTERS FOR REMOVING HEAT FROM SUBCUTANEOUS LIPID-RICH CELLS”;

U.S. Patent No. 8,603,073 entitled “SYSTEMS AND METHODS WITH INTERRUPT/RESUME CAPABILITIES FOR COOLING SUBCUTANEOUS LIPID-RICH CELLS”;

U.S. Pat. No. 8,192,474 entitled “TISSUE TREATMENT METHODS”;

U.S. Pat. No. 8,702,774 entitled “DEVICE, SYSTEM AND METHOD FOR REMOVING HEAT FROM SUBCUTANEOUS LIPID-RICH CELLS”;

U.S. Patent Publication No. 2012/0022518 entitled “COMBINED MODALITY TREATMENT SYSTEMS, METHODS AND APPARATUS FOR BODY CONTOURING APPLICATIONS”;

U.S. Pat. No. 9,314,368 entitled “HOME-USE APPLICATORS FOR NON-INVASIVELY REMOVING HEAT FROM SUBCUTANEOUS LIPID-RICH CELLS VIA PHASE CHANGE COOLANTS, AND ASSOCIATED DEVICES, SYSTEMS AND METHODS”;

U.S. Publication No. 2011/0238051 entitled “HOME-USE APPLICATORS FOR NON-INVASIVELY REMOVING HEAT FROM SUBCUTANEOUS LIPID-RICH CELLS VIA PHASE CHANGE COOLANTS, AND ASSOCIATED DEVICES, SYSTEMS AND METHODS”;

U.S. Publication No. 2012/0239123 entitled “DEVICES, APPLICATION SYSTEMS AND METHODS WITH LOCALIZED HEAT FLUX ZONES FOR REMOVING HEAT FROM SUBCUTANEOUS LIPID-RICH CELLS”;

U.S. Publication No. 2014/0277219 entitled “MULTI-MODALITY TREATMENT SYSTEMS, METHODS AND APPARATUS FOR ALTERING SUBCUTANEOUS LIPID-RICH TISSUE”; and

U.S. Publication No. 2014/0277302 entitled “TREATMENT SYSTEMS WITH FLUID MIXING SYSTEMS AND FLUID-COOLED APPLICATORS AND METHODS OF USING THE SAME”; and

U.S. Pat. No. 8,285,390 entitled “MONITORING THE COOLING OF SUBCUTANEOUS LIPID-RICH CELLS, SUCH AS THE COOLING OF ADIPOSE TISSUE.”

### TECHNICAL FIELD

The present disclosure relates generally to treatment systems, thermal devices, and methods for treating tissue. In particular, several embodiments are directed to transcutaneous systems, cooling devices, and methods for cooling nerves to inhibit muscle function.

### BACKGROUND

Habitual facial expressions can lead to permanent deep wrinkles, excessive expression lines, and other skin irregularities (e.g., frown lines, crow's-feet, worry lines, etc.) considered to be visually unappealing. They have proved to be vexing problems that are difficult to treat. Dynamic wrinkling of facial skin is caused by contractions of underlying facial muscles and can contribute to expression lines and deep wrinkles. Botulinum toxin therapy is often used to relax muscles to reduce dynamic wrinkling and to smooth skin. Botulinum toxin (e.g., Botox®) injected into facial tissue temporarily denervates or causes paralysis of facial muscles so that they are unable to contract. As such, dynamic wrinkles are reduced or eliminated for a period of time (e.g., 1 month to 4 months). Unfortunately, botulinum toxin injections can be painful, and a large number of injections may be needed to treat a desired area, and the botulinum toxin may spread and affect non-targeted surrounding tissue. Surgical procedures are also performed to treat wrinkles, sagging skin, and loose skin. These surgical procedures (e.g., face-lifts, brow-lifts, etc.) require long recovery times, lead to infections, and are generally irreversible. Accordingly, there is a need for more effective treatments of wrinkles and other conditions.

### BRIEF DESCRIPTION OF THE DRAWINGS

In the drawings, identical reference numbers identify similar elements or acts.

FIG. 1 is a schematic cross-sectional view of tissue with a non-invasive nerve cooling device in thermal contact with an exposed surface of the skin.

FIG. 2 is a partially schematic, isometric view of a treatment system for non-invasively affecting target regions of a subject in accordance with an embodiment of the technology.

FIG. 3 is a cross-sectional view of a connector taken along line 3-3 of FIG. 2.

FIG. 4 illustrates tissue that can be targeted in accordance with embodiments of the disclosure.

FIG. 5 is a flow diagram illustrating a method for improving the appearance of skin in accordance with embodiments of the technology.

FIG. 6 is a schematic block diagram illustrating computing system software modules and subcomponents of a computing device suitable to be used in treatment systems in accordance with embodiments of the technology.

### DETAILED DESCRIPTION clp A. Overview

The present disclosure describes treatment systems and methods for improving the appearance of a patient and other treatments. A method for treating a patient can include applying a non-invasive nerve cooling device to a target region along the patient's head and transcutaneously cooling nerve tissue to at least partially denervate a region to inhibit dynamic wrinkling. Several of the details set forth below are provided to describe the following examples and methods in a manner sufficient to enable a person skilled in the relevant art to practice, make, and use them. Several of the details and advantages described below, however, may not be necessary to practice certain examples and methods of the technology. Additionally, the technology may include other examples and methods that are within the scope of the technology but are not described in detail.

At least some embodiments are directed to reducing or eliminating dynamic wrinkles, lines (e.g., expression lines, frown lines, etc.), creases, and other skin irregularities considered to be visually unappealing. In some embodiments, nerve tissue can be injured to disrupt muscle function to, for example, inhibit muscle contractions that cause dynamic wrinkling. In some non-invasive procedures, nerve tissue can be transcutaneously cooled to cause a non-permanent injury to nerve tissue to at least partially block transmission of nervous system signals for a temporary amount of time (e.g., weeks or months). Advantageously, these procedures can be performed to improve cosmetic appearances while minimizing or limiting pain, risk of infections, and other problems associated with invasive procedures (e.g., injections, surgical procedures, etc.).

Various aspects of the technology are directed to cooling devices that cool/heat a target region for a period of time selected to localize thermal effects in targeted nerve tissue. The cooling devices can be controlled to inhibit or reduce injuries (e.g., permanent injuries, non-permanent injuries, etc.) to deeper tissue. The surface of the skin and/or targeted nerve tissue can be cooled to a temperature equal to or below about −40° C., −35° C., −30° C., −25° C., −20° C., −15° C., −10° C., −5° C., −2° C., or −1° C. for a treatment period equal to or longer than about 30 seconds, 1 minute, 2 minutes, 3 minutes, 4 minutes, 5 minutes, 7 minutes, 10 minutes, 12 minutes, 15 minutes, 30 minutes, or 1 hour. In some embodiments, a surface of the subject's skin at the target site is cooled to a low temperature (e.g., a temperature equal to or lower than about −20° C., about −10° C., about −5° C., about −2° C., or about −1° C.) to cause thermal injury to underlying nerve tissue. The cooling period can be shorter than about 30 seconds, or about 1, 2, 3, 4, 5, 7,10, 15, 20, 25, 30, 35, 40, 45, 50, or 55 minutes, or about 1 or 2 hours.

When treating the face, it is often undesirable to injure skin or deep muscle. In an extreme case, if the epidermis is overly frozen, hyperpigmentation (skin darkening) or hypopigmentation (skin lightening) can result, which is often undesirable. Cryoprotectants can be used to protect shallow tissue, such as the skin and in particular the epidermis, to avoid freezing and to avoid, minimize, or limit hyperpigmentation and/or hypopigmentation either immediately after the treatment or hours, a day, days, or weeks thereafter. The cooling device can have a temperature-controlled surface sized and configured to transcutaneously cool nerve tissue while minimizing or limiting cold injury to adjacent non-targeted tissue. The shape, configuration, thermal properties, and cooling capabilities of the cooling devices can be selected based on characteristics of the targeted tissue.

In addition to reducing dynamic wrinkling, treatment systems disclosed herein can cause skin tightening, thickening of tissue (e.g., thickening of the epidermis, dermis, and/or subcutaneous tissue), and/or a cold shock response at the cellular level. A treatment session can include performing different procedures to treat different tissue. For example, a first procedure can be performed to reduce dynamic wrinkling, and the same or different cooling device can be used to perform skin tightening procedures, skin thickening procedures, fat reducing procedures, or the like. In some embodiments, a treatment system has an applicator (e.g., a noninvasive tissue cooling device) configured to be applied to a subject's face to treat wrinkles around the eyes, mouth, forehead, etc. The applicator can injure nerve tissue to reduce dynamic wrinkling, reduce the size of wrinkles (e.g., depths, lengths, etc.), or the like. Conformable or contoured applicators can be applied to highly contoured regions around the eyes to reduce or eliminate, for example, crow's feet wrinkles. Treatment systems can also have applicators configured to be applied at other locations along the subject's body.

Some aspects of the technology are directed to treatment methods for cooling nerve tissue to affect the transmission of nervous system signals to muscles. In certain embodiments, damage to non-targeted tissue can be inhibited while producing a total or partial freeze event or zone. The freeze zone can be generally centered on a motor nerve to minimize or limit injuries to surrounding tissue. The severity and extent of freeze injury to the nerve tissue can be controlled to achieve the desired amount of denervation. For example, a sufficient amount of denervation can be achieved to inhibit contraction of one or more muscles so as to reduce or eliminate wrinkles (e.g., hyperdynamic wrinkles associated with habitual facial expressions). In some procedures, a single nerve branch can be injured to inhibit dynamic wrinkling at a specific location (e.g., forehead, brow, around the eyes, etc.). In other procedures, multiple nerve branches (e.g., temporal branch, zygomatic branch, etc.), a main nerve branch (e.g., temporofacial division of the facial nerve), or a nerve trunk can be injured to inhibit wrinkling at multiple locations or areas. For example, the temporofacial division of the facial nerve can be injured to block transmission of nervous system signals to the temporal and zygomatic branches and associated facial muscles. The number, locations, and severity of the injuries to the nervous system can be selected based on the desired areas (e.g., along face, neck, back, etc.) of denervation.

Some embodiments disclosed herein can be used for cosmetically beneficial alterations of a variety of regions. For example, some treatment procedures may be for the sole purpose of reducing or eliminating dynamic wrinkling or lines, or otherwise altering skin to conform to a cosmetically desirable look, feel, size, shape or other desirable cosmetic characteristic or feature. Accordingly, at least some embodiments of the cosmetic procedures can be performed without providing any therapeutic effect, or in another embodiment, providing minimal therapeutic effect. For example, skin treatment procedures can be performed without restoring of health, physical integrity, or the physical well-being of a subject.

Reference throughout this specification to “one example,” “an example,” “one embodiment,” or “an embodiment” means that a particular feature, structure, or characteristic described in connection with the example is included in at least one example of the present technology. Thus, the occurrences of the phrases “in one example,” “in an example,” “one embodiment,” or “an embodiment” in various places throughout this specification are not necessarily all referring to the same example. The headings provided herein are for convenience only and are not intended to limit or interpret the scope or meaning of the technology.

### B. Cryotherapy

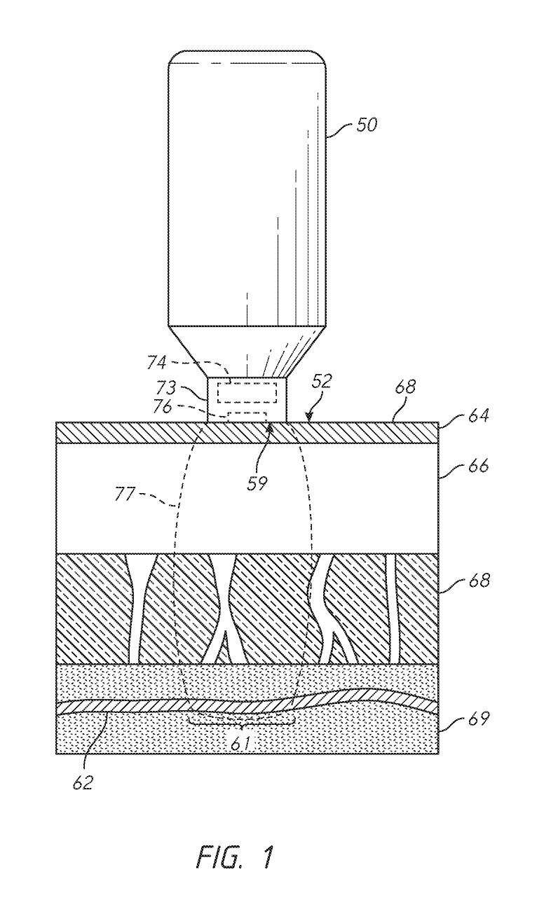

FIG. 1 is a schematic cross-sectional view of tissue with a non-invasive nerve cooling device 50 (“cooling device 50”) that cools targeted nerve tissue 61 of a nerve 62 located below the epidermis 64 and dermis 66 layers of skin 68. The cooling device 50 can transcutaneously cool the nerve tissue 61 so as to produce a sufficient injury to inhibit transmission of nervous system signals to muscles that contribute to wrinkles, expression lines, or other unwanted features. The nerve tissue 61 can be part of nerve trunks, nerve branches, and other sections of the nervous system and may be located in subcutaneous tissue 69.

The cooling device 50 can produce a cooling zone 77 (shown in phantom line) of tissue at or below a target temperature. The depth of the cooling zone 77 can be selected to avoid injuring deeper non-targeted tissue. In one procedure, the cooling zone 77 comprises most of the tissue directly between the targeted nerve tissue 61 and the skin surface 52. Adjacent tissue may also be cooled but can be at a sufficiently high temperature to avoid thermal injury. As such, nerve tissue 61 can be injured while adjacent muscle, connective tissue, etc., is not injured, or is injured to a lesser extent, because surrounding tissue may be less prone to cold injury than the nerve tissue 61. Also, when the cooling device 50 is centered over a nerve, adjacent, laterally offset tissue typically can be cooled to a lesser extent than the nerve tissue directly beneath the cooling device.

A thermal element 73 of the cooling device 50 can include one or more thermal features 74 that can include, without limitation, thermoelectric elements, fluid channels through which coolant flows, resistive heaters, energy emitters, and/or other elements capable of heating and/or cooling. In thermoelectric embodiments, the thermal element 73 includes a heat-exchanging plate, Peltier device(s) 74 (e.g., a single Peltier element, an array of Peltier elements, etc.), or the like. In non-thermoelectric embodiments, the features 73 can be fluid channels for cooling/heating using only temperature-controlled liquid.

The thermal element 73 can have a temperature-controlled exposed surface 59. The surface 59 can be part of a heat-exchanging plate. In another embodiment, the surface 59 can be the surface of an interface layer or a dielectric layer. The area of the surface 59 can be equal to or smaller than 0.5 cm², 1 cm², 2 cm², 3 cm², 4 cm², or 5 cm² to limit the size (e.g., width, depth, etc.) of the cooling zone 77. The temperature-controlled surface 59 can have a polygonal shape, a circular shape, an elongated shape (e.g., elliptical shape), or other shape selected to provide the desired cooling zone. For example, the surface 59 can be elongated and generally aligned with the direction of the nerve 62 such that the cooling zone 77 is generally aligned with a section of the nerve 62.

The thermal element 73 can also include one or more sensors 76 configured to identify tissue, anatomical features, measure tissue impedance, applied force, and/or tissue contact. In some embodiments, the sensor 76 can be an electrical, optical, or mechanical nerve detection sensor. The number and types of sensors can be selected based on the location and characteristics of the targeted nerve tissue.

Nerve cells transmit electrical impulses, and nerve fibers are prolonged axons that conduct electrical impulses. The electrical impulses are converted to chemical signals to communicate with cells, such as effector cells or other nerve cells. Structures of nerves can be injured to attenuate one or more nervous system signals transmitted by nerve tissue. Without being bound by theory, the effect of cooling is believed to result in, for example, injury, damage, disruption, shrinkage, disabling, damaging, destroying, removing, killing, or other methods of tissue alteration. Such alteration is believed to stem from one or more mechanisms acting alone or in combination. Cooling denervation can include affecting (e.g., injuring, damaging, etc.) most or all of the nerve tissue along a section of a nerve (e.g., nerve trunk or branch) to stop substantially all the signals from traveling to more distal locations along the nervous system. When the signals are attenuated (e.g., reduced, cut off, etc.), muscles can relax to reduce or eliminate dynamic wrinkling. For example, a cold injury can at least partially block nerve signals to minimize or limit the appearance of hyperdynamic facial wrinkles, frown lines, crow's-feet, and other facial wrinkles.

The effect of cooling nerves is to selectively injure, damage, or otherwise affect nerve tissue. In some procedures, the cooling device 50 can cool the exposed skin surface to a temperature in a range from about −50° C. to about 10° C., about −40° C. to about −2° C., about −25° C. to about −5° C., about −20° C. to about −5° C., or other suitable temperature ranges. The treatment site can be cooled/heated any number of times in different sequences selected based on the procedure to be performed. Periods of heating/cooling can be equal to or less than about 1 minute, 2 minutes, 3 minutes, 4 minutes, 5 minutes, 10 minutes, 20 minutes, 30 minutes, 50 minutes, 1 hour, 70 minutes, 2 hours, etc. In one procedure, nerve tissue is continuously or intermittently cooled for a cooling period, and then continuously or intermittently heated for a heating period.

One expected advantage of the foregoing techniques is that nerve tissue (e.g., cells) in the target region can be selectively injured due to nerve structures being generally more susceptible to cold injury than other tissue. This difference in sensitivity allows the nerve injury without collateral permanent damage to non-nerve tissue in the same region. The cold injury to the nerve tissue can be a non-freezing injury or a freezing injury. During procedures that require sustained exposure to cold temperatures, methods of protecting the overlying tissue (e.g., typically water-rich dermal and epidermal skin cells overlying the target nerve) from freeze damage may include improving the freeze tolerance and/or freeze avoidance of these cells using cryoprotectant applied to zones where freeze protection is desired. Cryoprotectants can be topically applied to inhibit or prevent freeze damage of the tissue between the cooled surface 59 and the targeted nerve tissue 61.

In some procedures, a protected region in which a majority of tissue between the skin area contacted by the cooling device 50 and the injured nerve tissue is not injured by the transcutaneously cooling can be protected using cryoprotectant. For example, the protected region can include most of the thickness of the skin directly between the nerve tissue 61 (e.g., a peripheral nerve, a sensory nerve, or a motor nerve) and area of skin contacted by the surface 59.

### C. Treatment Systems

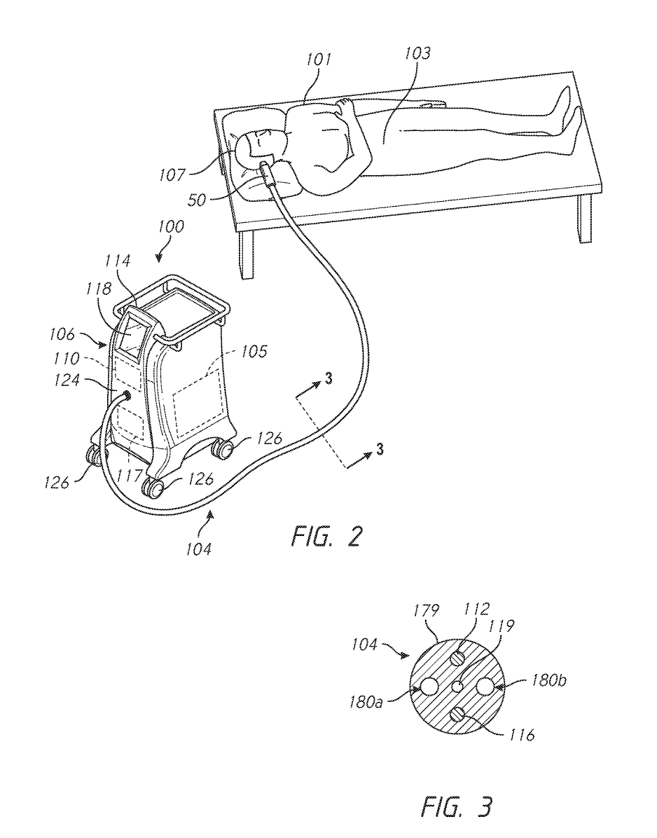

FIG. 2 shows the treatment system 100 that includes the cooling device 50, a connector 104, and a control module 106. The cooling device 50 is positioned to injure nerve tissue along a patient's temporal region. The restraining means 107 can hold the cooling device 50 against a subject 103 (e.g., an adult human) and can be, for example, a strap system, a helmet, or other suitable apparatus for holding the cooling device 50. The connector 104 can provide energy (e.g., electrical energy) and fluid (e.g., coolant) from the control module 106 to the cooling device 50. An operator can use the control module 106 to control operation of the cooling device 50.

FIG. 3 is a cross-sectional view of the connector 104 taken along line 3-3 of FIG. 2 in accordance with at least some embodiments of the technology. The connector 104 can include a main body 179, a supply fluid line or lumen 180a (“supply fluid line 180a”), and a return fluid line or lumen 180b (“return fluid line 180b”). The main body 179 may be configured (via one or more adjustable joints) to “set” in place for the treatment of the subject 101. The supply and return fluid lines 180a, 180b can be conduits comprising, in whole or in part, polyethylene, polyvinyl chloride, polyurethane, and/or other materials that can accommodate circulating coolant, such as water, glycol, synthetic heat transfer fluid, oil, refrigerant, and/or any other suitable heat-conducting fluid. In one embodiment, each fluid line 180a, 180b can be a flexible hose surrounded by the main body 179. The connector 104 can also include one or more electrical lines 112 for providing power to the cooling device 50 and one or more control lines 116 for providing communication between the control module 106 (FIG. 2) and the cooling device 50 (FIGS. 1 and 2). In various embodiments, the connector 104 can include a bundle of fluid conduits, a bundle of power lines, wired connections, and other bundled and/or unbundled components selected to provide ergonomic comfort, minimize unwanted motion (and thus potential inefficient removal of heat from the subject 101), and/or to provide an aesthetic appearance to the treatment system 100.

Referring again to FIG. 2, the control module 106 can include a fluid chamber or reservoir 105 (illustrated in phantom line), a power supply 110 (illustrated in phantom line), and a controller 114 carried by a housing 124 with wheels 126. The control module 106 can include a refrigeration unit, a cooling tower, a thermoelectric chiller, heaters, or any other device capable of controlling the temperature of coolant in the fluid chamber 105. The coolant can be continuously or intermittently delivered to the cooling device 50 via the supply fluid line 180a (FIG. 3) and can circulate through the cooling device 50 to absorb heat. The coolant, which has absorbed heat, can flow from the cooling device 50 back to the control module 106 via the return fluid line 180b (FIG. 3). For warming periods, the control module 106 can heat the coolant such that warm coolant is circulated through the cooling device 50. Alternatively, a municipal water supply (e.g., tap water) can be used in place of or in conjunction with the control module 106.

In some embodiments, the cooling device 50 can provide a vacuum to hold the subject's skin against the cooled surface (surface 59 in FIG. 1) and minimize blood flow in the region being cooled. A pressurization device 117 can provide suction via a vacuum line 119 (FIG. 3) and can include one or more pumps. Air pressure can either be controlled with a regulator between the pressurization device 117 and the cooling device 50, or pressure may be reduced up to the maximum capacity of the pressurization device 117. In other embodiments, the cooling device 50 may not provide any vacuum.

An operator can control operation of the treatment system 100 using an input/output device 118 of the controller 114. The input/output device 118 can display the status of the procedure (e.g., percentage of procedure completed), state of operation of the cooling device 50, or other information. The power supply 110 can provide a direct current voltage for powering electrical elements of the cooling device 50 via the line 112 (FIG. 3). In some embodiments, the controller 114 can exchange data with the cooling device 50 via a wireless or an optical communication link and can monitor and adjust treatment based on one or more treatment profiles and/or patient-specific treatment plans, such as those described, for example, in commonly assigned U.S. Pat. No. 8,275,442. Each treatment profile can include one or more segments, and each segment can include specified durations (e.g., 1 minute, 2 minutes, 3 minutes, 4 minutes, 5 minutes, 7 minutes, 10 minutes, 15 minutes, 20 minutes, 30 minutes, 1 hour, 2 hours, etc.), a target profile, etc. Treatment profiles can be selected based upon the targeted treatment site. For example, treatment profiles to injure temporal nerve tissue may be different from treatment profiles for injuring nerve tissue at other locations. Tissue injury can be controlled by adjusting thermal parameters, including (1) cooling rate, (2) end (e.g., minimum) temperature, (3) time held at the minimum temperature (e.g., hold time), (4) temperature profile, and (5) warming or thawing rates.

### D. Methods of Treatment

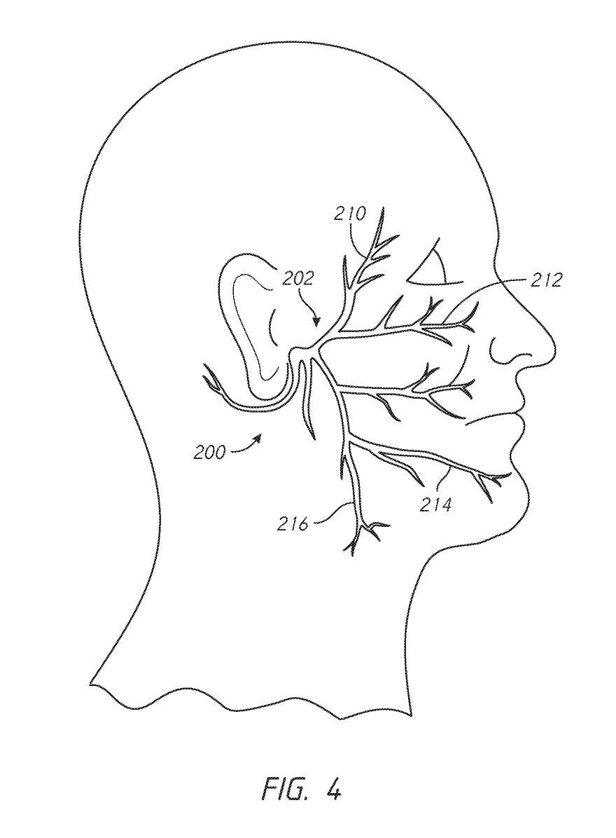

FIG. 4 illustrates nerve tissue that can be targeted in accordance with embodiments of the disclosure. A facial nerve 200 is the seventh cranial nerve and is the motor nerve for muscles in the face. A temporal region 202 of the facial nerve 200 can be targeted to affect the temporal branch 210, zygomatic branch 212, mandibular branch 214, and/or cervical branch 216. Cooling can be controlled to localize injuries to one or more of these branches.

The corrugator supercilii muscle can contract to draw the eyebrows downward and inward and lead to vertical dynamic wrinkles in the forehead and in the space between the eyebrows. The temporal branch 210 can be injured to inhibit contraction of or otherwise affect the corrugator supercilii muscle. The procerus muscle pulls down the inner portions of the eyebrows to reduce transverse dynamic wrinkles over the bridge of the nose. The zygomatic branch 212 can be injured to inhibit contraction of the procerus muscle. Platysma myoides muscle produce dynamic wrinkles along the surface of the neck. The cervical branch 216 can be injured to inhibit contraction of or otherwise affect the platysma myoides muscle. Nerve tissue at other locations can also be treated.

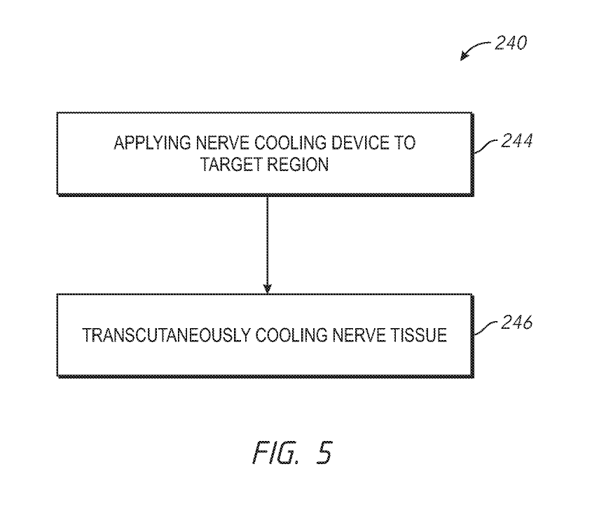

FIG. 5 is a flow diagram illustrating a method 240 for improving the appearance of a subject in accordance with embodiments of the disclosure. Generally, an early stage of the method 240 can include applying a non-invasive nerve cooling device to the surface of the subject's skin. To treat facial wrinkles, the cooling device can be placed along a facial region to target nerve tissue discussed in connection with FIG. 4. Details of the method 240 are discussed below.

At block 244, the cooling device is applied to the target region. Treatment regions can be identified by locating nerves using ultrasound energy, electrical energy, landmark imaging, or other locating techniques. For example, cooling device 50 (FIG. 1) can have nerve detection sensors with energy emitters for outputting energy (e.g., ultrasound energy, electrical energy, etc.) suitable for locating nerve tissue, landmarks (e.g., bones), or other anatomical features. Tissue detection techniques can be selected based on the location of the treatment site.

At block 246, the cooling device cools nerve tissue until the nerve tissue is injured. The length of time the nerve tissue is kept at or below the target temperature can be selected based on the desired severity of the injury. Motor neurons, axons, and innervated muscle fibers can be injured to affect the motor unit of the neuromuscular system. In some procedures, nervous system impulses are inhibited (e.g., disrupted, blocked, etc.) by injuring nerve fibers (e.g., motor fibers, sensor fibers, etc.), motor endplates, or other nerve structures.

The targeted nerve tissue can be sufficiently injured to inhibit facial muscle contractions and thereby reduce facial wrinkles, expression lines, or other undesired skin irregularities. In some embodiments, the cooling device can cool nerve tissue to injure the nerve tissue so as to at least partially block transmission of nervous system signals to facial muscles. The amount of injured nerve tissue and/or the extent of injury can be selected to achieve the desired blocking. For example, the amount of nerve tissue that is injured can be increased or decreased by increasing or decreasing the size of the cooling zone (e.g., cooling zone 77 of FIG. 1) and/or increasing or decreasing the number of treatment sites. The nerve tissue can be injured without damaging to any significant extent muscle underlying and/or overlying the nerve tissue to maintain continued normal function of adjacent muscle. For example, nerve tissue can be cooled for a sufficient length of time to cause a desired thermal injury selectively to nerve cells. The cooling period can be sufficiently short to minimize or limit thermal injury to the surrounding tissue.

Tissue can be supercooled so as to not create any partial or total freeze event. Alternatively, a partial or total freeze event in a cooling zone (e.g., cooling zone 77 of FIG. 1) can be maintained by continuously or periodically cooling the patient's tissue to keep a target volume of nerve tissue frozen for a period of time long enough to sufficiently affect nerve function. Referring to FIG. 1, cryoprotectant can be used to inhibit or prevent freezing of the epidermis 64, dermis 66, and/or connective tissue to localize the freeze event to the nerve tissue 61 and, in some procedures, a region of the muscle 69 underlying and/or overlying the nerve tissue 61.

The treatment site can be periodically or continuously monitored using the sensor 76 of FIG. 1. The sensor 76 can be an optical sensor capable of detecting changes in the optical characteristics of tissue caused by treatment. Freezing of tissue can cause such optical changes. The optical sensor 76 can include one or more energy emitters (e.g., light sources, light emitting diodes, etc.), detector elements (e.g., light detectors), or other components for non-invasively monitoring optical characteristics of tissue. In place of or in conjunction with monitoring using optical techniques, tissue can be monitored using electrical and/or mechanical techniques because changes in electrical impedance and/or mechanical properties of the tissue can be detected and may indicate tissue changes.

In some procedures, nerve tissue can experience a supercooling event or a freeze event that causes non-permanent injuries to block the transmission of nervous system signals for a period of time. The period of time can be equal to or longer than about 1 day, 1 week, 1 month, multiple months, (e.g., 3 months, 4 months, 5 months, etc.), or other desired time period. For example, facial nerve tissue can be sufficiently injured to inhibit facial muscle contractions for at least about 1 day, 1 week, multiple weeks, 1 month, multiple months (e.g., 3-4 months), or longer. In one procedure, the temporal branch and the zygomatic branch are concurrently or sequentially injured to inhibit facial muscular contractions along the subject's forehead and around the subject's eyes for at least 1 month, 2 months, or other suitable time periods. The severity of the injury can be increased or decreased to increase or decrease recovery times.

Over time, the effects of the procedure can diminish. Damaged nerves will regenerate and heal. The patient can be evaluated to determine whether to perform additional procedures at suitable intervals. Wrinkles (e.g., dynamic wrinkles) can be evaluated and measured using different tests, including a 5-point wrinkle scale or a 9-point global improvement scale; or other suitable scales or tests can be used to monitor the effect of the treatment over long periods of time. Additional procedures can be performed to prevent or inhibit the reoccurrence of wrinkling.

A substance can be applied to the subject's skin before applying the cooling device at block 244 of FIG. 5. The substance can be used to (a) provide thermal coupling between the subject's skin and cooling devices (e.g., cooling plates of cooling devices) to improve heat transfer therebetween, (b) selectively protect non-target tissues from freeze damage (e.g., damage due to crystallization), and/or (c) promote freeze events by increasing nucleation sites. The substance may be a fluid, a gel, or a paste and may be hygroscopic, thermally conductive, and biocompatible. In some embodiments, the substance can be a cryoprotectant that reduces or inhibits cell destruction. As used herein, “cryoprotectant,” “cryoprotectant agent,” and “composition” mean substances (e.g., compositions, formulations, compounds, etc.) that assist in preventing freezing of tissue compared to an absence of the substances(s). In one embodiment, the cryoprotectant allows, for example, the cooling device to be pre-cooled prior to being applied to the subject for more efficient treatment. Further, the cryoprotectant can also enable the device to be maintained at a desired low temperature while preventing ice from forming on a surface (e.g., heat-exchanging surface 59 of FIG. 1), and thus can reduce the delay in reapplying the device to the subject. Yet another aspect of the technology is that the cryoprotectant may prevent the treatment device from freezing to the skin of the patient or subject. Additionally or alternatively, the cryoprotectant can allow microscopic crystals to form in the tissue but can limit crystal growth that would cause cell destruction, and in some embodiments, the cryoprotectant can allow for enhanced uptake or absorption and/or retention in target tissue prior to and during the introduction of cooling.

Suitable cryoprotectants and processes for implementing cryoprotectants are described in commonly assigned U.S. Patent Publication No. 2007/0255362. The cryoprotectant may additionally include a thickening agent, a pH buffer, a humectant, a surfactant, and/or other additives and adjuvants as described herein. Freezing point depressants may include, for example, propylene glycol (PG), polyethylene glycol (PEG), dimethyl sulfoxide (DMSO), or other suitable alcohol compounds. In a particular embodiment, a cryoprotectant may include propylene glycol, glycerin (a humectant), and ethanol. In another embodiment, a cryoprotectant may include propylene glycol, hydroxyethyl cellulose (a thickening agent), and water. In a further embodiment, a cryoprotectant may include polypropylene glycol, glycerin, and ethanol. The freezing point depressant may also include ethanol, propanol, iso-propanol, butanol, and/or other suitable alcohol compounds. Certain freezing point depressants (e.g., PG, PPG, PEG, etc.) may also be used to improve spreadability of the cryoprotectant and to provide lubrication. The freezing point depressant may lower the freezing point of tissue and/or body liquids/lipids to about 0° C. to −50° C., about 0° C. to −50° C., or about 0° C. to −30° C. In other embodiments, the freezing point of the liquids/lipids can be lowered to about −10° C. to about −40° C., about −10° C. to about −30° C., or about −10° C. to about −20° C. In certain embodiments, the freezing point of the liquids/lipids can be lowered to a temperature below about 0° C., −5° C., −10° C., −12° C., −15° C., −20° C., −30° C., or −35° C.

### E. Suitable Computing Environments

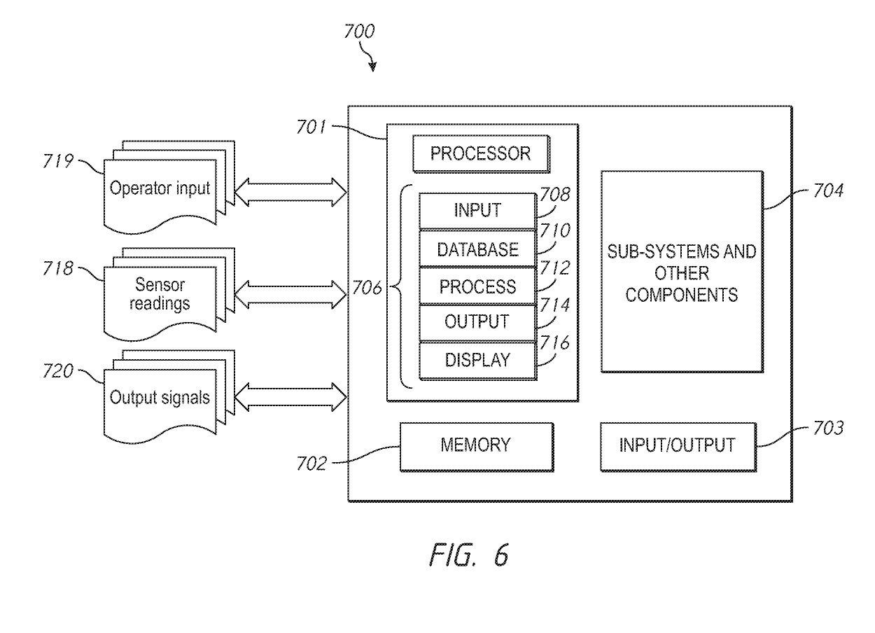

FIG. 6 is a schematic block diagram illustrating subcomponents of a controller in accordance with an embodiment of the disclosure. A controller or computing device 700 can be the controller 114 of FIG. 2 or can be incorporated into the applicators (e.g., cooling device 50 of FIG. 1) disclosed herein. The controller 700 can include a computing device having a processor 701, a memory 702, input/output devices 703, and/or subsystems and other components 704. The computing device 700 can perform any of a wide variety of computing processing, storage, sensing, imaging, and/or other functions. Components of the computing device 700 may be housed in a single unit or distributed over multiple, interconnected units (e.g., through a communications network). The components of the computing device 700 can accordingly include local and/or remote memory storage devices and any of a wide variety of computer-readable media.

As illustrated in FIG. 6, the processor 701 can include a plurality of functional modules 706, such as software modules, for execution by the processor 701. The various implementations of source code (i.e., in a conventional programming language) can be stored on a computer-readable storage medium or can be embodied on a transmission medium in a carrier wave. The modules 706 of the processor can include an input module 708, a database module 710, a process module 712, an output module 714, and, optionally, a display module 716.

In operation, the input module 708 accepts an operator input 719 (e.g., characteristics of wrinkles, location of wrinkles, etc.) via the one or more input devices, and communicates the accepted information or selections to other components for further processing. The database module 710 organizes records, including patient records, treatment data sets, treatment profiles and operating records, and other operator activities; and it facilitates storing and retrieving of these records to and from a data storage device (e.g., internal memory 702, an external database, etc.). Any type of database organization can be utilized, including a flat file system, hierarchical database, relational database, distributed database, etc.

In the illustrated example, the process module 712 can generate control variables based on sensor readings 718 from sensors (e.g., sensors 76 of FIG. 1) and/or other data sources, and the output module 714 can communicate operator input to external computing devices and control variables to the controller. The display module 716 can be configured to convert and transmit processing parameters, sensor readings 718, output signals 720, input data, treatment profiles and prescribed operational parameters through one or more connected display devices, such as a display screen, printer, speaker system, etc.

In various embodiments, the processor 701 can be a standard central processing unit or a secure processor. Secure processors can be special-purpose processors (e.g., a reduced instruction set processor) that can withstand sophisticated attacks that attempt to extract data or programming logic. The secure processors may not have debugging pins that enable an external debugger to monitor the secure processor's execution or registers. In other embodiments, the system may employ a secure field programmable gate array, a smartcard, or other secure devices.

The memory 702 can be standard memory, secure memory, or a combination of both memory types. By employing a secure processor and/or secure memory, the system can ensure that data and instructions are highly secure and that sensitive operations such as decryption are shielded from observation. In various embodiments, the memory 702 can be flash memory, secure serial EEPROM, a secure field programmable gate array, or a secure application-specific integrated circuit.

The input/output device 118 can include, without limitation, a keyboard, a mouse, a stylus, a push button, a switch, a potentiometer, a scanner, an audio component such as a microphone, or any other device suitable for accepting user input, and can also include one or more video monitors, medium readers, audio devices such as a speaker, any combination thereof, and any other device or devices suitable for providing user feedback. For example, if the cooling device 50 moves an undesirable amount during a treatment session, the input/output device 703 can alert the subject 101 and/or operator via an audible alarm. The input/output device (e.g., input/output device 118 of FIG. 2) can be a touch screen that functions as both an input device and an output device. The control panel can include visual indicator devices or controls (e.g., indicator lights, numerical displays, etc.) and/or audio indicator devices or controls. The control panel may be a component separate from the input and/or output device, may be integrated with one or more of the devices, may be partially integrated with one or more of the devices, may be in another location, and so on. In alternative embodiments, the controller can be contained in, attached to, or integrated with the cooling devices and applicators disclosed herein. In yet other embodiments, the various components can be fixedly installed at a treatment site. Further details with respect to components and/or operation of applicators, control modules (e.g., treatment units), and other components may be found in commonly assigned U.S. Patent Publication No. 2008/0287839.

The controller 700 can include any processor, Programmable Logic Controller, Distributed Control System, secure processor, and the like. A secure processor can be implemented as an integrated circuit with access-controlled physical interfaces, tamper resistant containment, means of detecting and responding to physical tampering, secure storage, and shielded execution of computer-executable instructions. Some secure processors also provide cryptographic accelerator circuitry. Suitable computing environments and other computing devices and user interfaces are described in commonly assigned U.S. Pat. No. 8,275,442, entitled “TREATMENT PLANNING SYSTEMS AND METHODS FOR BODY CONTOURING APPLICATIONS,” which is incorporated herein in its entirety by reference.

The controller 700 can store, determine, and/or monitor thermal cycles for sequentially cooling and heating a treatment site any number of times. The controller 700 can select the order and lengths of thermal cycles (e.g., heating cycles, cooling cycles, etc.), target parameters (e.g., temperatures, temperature ranges, etc.), and/or temperature profiles. After cooling, cooling devices can be actively or passively warmed to room temperature, skin temperature, or another suitable temperature. For example, the thermoelectric elements of the cooling devices can be passively (e.g., naturally) returned to room temperature prior to removing the applicator from the subject.

The applicators in some embodiments can deliver energy (e.g., radiofrequency energy, ultrasound energy, etc.) to and remove heat from the target region. A session may have a single stage of delivering energy that ceases prior to a single stage of removing heat from target nerve tissue. Additionally, sequential application of the stages of heating or cooling may occur multiple times so that multiple non-overlapping stages of energy delivery and heat removal occur. For example, thermal elements of an applicator can perform a heating cycle while other thermal elements of the applicator perform a cooling cycle. The controller 700 can store various executable programs for controlling applicators disclosed herein to perform a wide range of thermal cycles for body contouring, treating cellulite, improving skin appearance, targeting glands, and/or performing other methods as described in, for example, U.S. patent application Ser. No. 14/611,127 entitled “TREATMENT SYSTEMS, METHODS, AND APPARATUS FOR IMPROVING THE APPEARANCE OF SKIN AND PROVIDING FOR OTHER TREATMENTS”, U.S. patent application Ser. No. 14/611,052 entitled “TREATMENT SYSTEMS AND METHODS FOR TREATING CELLULITE AND FOR PROVIDING OTHER TREATMENTS,” and International Patent Application No. PCT/US2015/013,971 entitled “TREATMENT SYSTEMS AND METHODS FOR AFFECTING GLANDS AND OTHER TARGETED STRUCTURES,” which are incorporated herein in their entireties by reference.

Different types of cooling techniques can be used to thermally affect targeted nerve tissue. For example, treatment systems and devices are disclosed herein to control thermal parameters such that nerve tissue/body fluids within the treatment site are supercooled to temperatures below the freezing point without forming or nucleating ice crystals so that a non-freezing treatment results. Alternatively or additionally, after a supercooling state exists, the supercooled tissue/body fluids can then be intentionally nucleated to create a freeze zone and damage, reduce, disrupt, or otherwise affect the targeted cells. Nucleation can be induced by delivering an alternating current to the tissue, applying a nucleating solution onto the surface of the skin (e.g., one that includes bacteria which initiate nucleation), and/or by creating a mechanical perturbation to the tissue, such as by use of vibration, ultrasound energy, etc. In some procedures, the surface of the subject's skin can be cooled to create a supercooled cooling zone that includes the target nerve tissue. The surface of the subject's skin can then be heated to warm non-targeted shallow tissue while the nerve tissue and/or surrounding body fluid remain in supercooled states. Nucleation can then be induced in the localized supercooled region without substantially freezing or altering the warmed shallow tissue. The controller 700 can store various executable programs for controlling applicators disclosed herein to perform these techniques.

### F. Conclusion

Various embodiments of the technology are described above. It will be appreciated that details set forth above are provided to describe the embodiments in a manner sufficient to enable a person skilled in the relevant art to make and use the disclosed embodiments. Furthermore, features, structures, or characteristics of various embodiments may be combined in any suitable manner. Moreover, one skilled in the art will recognize that there are a number of other technologies that could be used to perform functions similar to those described above. While processes or blocks are presented in a given order, alternative embodiments may perform routines having stages, or employ systems having blocks, in a different order, and some processes or blocks may be deleted, moved, added, subdivided, combined, and/or modified. Each of these processes or blocks may be implemented in a variety of different ways. Also, while processes or blocks are at times shown as being performed in series, these processes or blocks may instead be performed in parallel, or may be performed at different times. The headings provided herein are for convenience only and should not be used to interpret the scope or meaning of the described technology.

Unless the context clearly requires otherwise, throughout the description, the words “comprise,” “comprising,” and the like are to be construed in an inclusive sense, as opposed to an exclusive or exhaustive sense; that is to say, in a sense of “including, but not limited to.” Words using the singular or plural number also include the plural or singular number, respectively. Use of the word “or” in reference to a list of two or more items covers all of the following interpretations of the word: any of the items in the list, all of the items in the list, and any combination of the items in the list. Furthermore, the phrase “at least one of A, B, and C, etc.” is intended in the sense that one having ordinary skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense that one having ordinary skill in the art would understand the convention (e.g., “a system having at least one of A, B, or C” would include but not be limited to systems that have A alone, B alone, C alone, A and B together, A and C together, B and C together, and/or A, B, and C together, etc.).

Any patents, applications and other references, including any that may be listed in accompanying filing papers, are incorporated herein by reference. Aspects of the described technology can be modified, if necessary, to employ the systems, functions, and concepts of the various references described above to provide yet further embodiments. These and other changes can be made in light of the above Detailed Description. While the above description lists certain embodiments and describes the best mode contemplated, no matter how detailed the description, various changes can be made. Implementation details may vary considerably, while still being encompassed by the technology disclosed herein. As noted above, particular terminology used when describing certain features or aspects of the technology should not be taken to imply that the terminology is being redefined herein to be restricted to any specific characteristics, features, or aspects of the technology with which that terminology is associated. The various aspects and embodiments disclosed herein are for purposes of illustration and are not intended to be limiting, with the true scope and spirit being indicated by the following claims.

What is claimed is:

1. A method for treating a subject, comprising:
applying a non-invasive nerve cooling device to a target region along the subject's head; and
transcutaneously cooling nerve tissue at the target region using the non-invasive nerve cooling device to injure the nerve tissue to inhibit facial muscular contractions and thereby reduce dynamic wrinkles and/or expression lines.

2. The method of claim 1, wherein transcutaneously cooling the nerve tissue causes non-permanent injury to the nerve tissue to at least temporarily partially block transmission of nervous system signals to the subject's facial muscles.

3. The method of claim 1, wherein transcutaneously cooling the nerve tissue includes injuring the nerve tissue without damaging, to any significant extent, facial muscle associated with the nerve tissue.

4. The method of claim 1, wherein transcutaneously cooling the nerve tissue includes cooling a surface of the subject's skin at the target site to a temperature equal to or lower than about −2 degrees Celsius.

5. The method of claim 1, wherein transcutaneously cooling the nerve tissue includes non-permanently injuring a sufficient amount of the nerve tissue to inhibit muscle contractions along the subject's forehead and/or around the subject's eyes.

6. The method of claim 1, further comprising repeatedly applying the non-invasive nerve cooling device to the subject to injure nerve tissue at different locations along a nerve branch to at least partially block transmission of nervous system signals to overactive facial expression muscles.

7. The method of claim 1, wherein the nerve tissue is sufficiently injured to inhibit facial muscle contractions for at least about 1 week.

8. The method of claim 1, wherein injuring the nerve tissue includes injuring nerves that control facial expression.

9. The method of claim 1, further comprising delivering a cryoprotectant to a surface of the patient's skin to inhibit injury of non-targeted tissue.

10. The method of claim 1, further comprising:
determining the location of the nerve tissue using ultrasound energy, electrical energy, and/or landmark imaging.

11. The method of claim 1, wherein the nerve tissue is thermally injured due to freezing.

12. A system for treating a subject, comprising:
a non-invasive nerve cooling device configured to be applied to an external surface of the subject's skin; and
a controller programmed to cause the non-invasive nerve cooling device to perform the method in claim 1.

13. A system for affecting a treatment area, comprising:
a non-invasive nerve cooling device configured to cool nerve tissue of a subject; and
a controller having instructions for causing the non-invasive nerve cooling device to cool a surface of the subject's skin for a cooling period such that the nerve tissue is injured to inhibit muscular contractions so as to reduce the appearance of facial wrinkles and/or expression lines after the cooling period ends.

14. The system of claim 13, wherein the controller has instructions to cause the system to monitor the treatment area while the non-invasive nerve cooling device is applied to the treatment area and to control operation of the non-invasive nerve cooling device based on the monitoring such that at least a portion of the nerve tissue is at or below a target temperature for the cooling period.

15. The system of claim 13, wherein the non-invasive nerve cooling device includes one or more nerve detection sensors.

16. The system of claim 13, wherein the cooling period is about 30 seconds to about 1 hour.

17. The system of claim 13, wherein the cooling period is shorter than about 1, 2, 3, 4, 5, 7, 10, 12, 15, 20, 25, 30, 35, 40, 45, 50, or 55 minutes or about 2 hours.

18. The system of claim 13, wherein the non-invasive nerve cooling device has a temperature-controlled surface for being applied to the treatment area, and wherein an area of the temperature-controlled surface is less than about 2 cm².
